(19) United States
(12) Patent Application Publication
(10) Pub. No.: US 2016/0200043 A1
(43) Pub. Date: Jul. 14, 2016

(54) 3-DIMENSIONAL BIOSCAFFOLDS

(71) Applicant: NATIONAL UNIVERSITY OF SINGAPORE, Singapore (SG)

(72) Inventors: Eng San Thian, Singapore (SG); Ying Hsi Jerry Fuh, Singapore (SG); Jie Sun, Singapore (SG); En Jen Wilson Wang, Singapore (SG); Geok Soon Hong, Singapore (SG); Yoke San Wong, Singapore (SG); Jinlan Li, Singapore (SG); Yilin Guo, Singapore (SG)

(73) Assignee: National University of Singapore, Singapore (SG)

(21) Appl. No.: 14/913,430

(22) PCT Filed: Aug. 25, 2014
(86) PCT No.: PCT/SG2014/000396

§ 371 Date: Feb. 22, 2016

(30) Foreign Application Priority Data
Aug. 23, 2013 (GB) 1315074.3

(51) Int. Cl. B29C 67/00; D01D 5/00; A61F 2/02

(52) U.S. Cl. CPC B29C 67/0055; A61F 2/02; B29C 67/0085; D01D 5/003; A61F 2/30756

(57) ABSTRACT

The invention concerns an apparatus and a method for the manufacture of a three-dimensional (3D) bioscaffold; a 3D bioscaffold made using same; and the use of said 3D bioscaffold in the manufacture of an implant to treat injuries such as, but not limited to, meniscal injuries.

### FIELD OF THE INVENTION

The invention concerns an apparatus and a method for the manufacture of a three-dimensional (3D) bioscaffold; a 3D bioscaffold made using said method; and the use of said 3D bioscaffold in the manufacture of an implant to treat injuries such as, but not limited to, meniscal injuries.

### BACKGROUND OF THE INVENTION

The menisci of the knee are two semilunar fibrocartilage discs, located in the knee joint between tibia and femur, improving stability and aiding rotatory movements of the knee, acting as a shock absorber and providing nutrition in the form of synovial fluid to the articular cartilage. In humans meniscal structures are not just found in the knee joint, but are also present in acromioclavicular, sternoclavicular and temporomandibular joints.

The meniscus is typically an avascular structure with the primary blood supply limited to the periphery, the inner part of the meniscus is nourished by the synovial fluid through diffusion. The restricted blood supply hinders the regeneration capacity of the fibro-cartilagenous tissue, resulting in a long and often incomplete healing process, which often requires surgery to remove or replace damaged tissue.

Meniscus lesions are among the most frequent injuries in orthopaedic practice and can be the result of a trauma or twisting of the knee. Athletes, particularly those who play contact sports, are at risk of meniscal tears. However, damage to the meniscus can also occur when tissue is weakened as through age in the elderly.

Current methods for treatment is arthroscopic meniscectomy; a minimally invasive surgical procedure to remove all or part of a damaged meniscus in the knee or, if possible, to repair a meniscus. However, the removal of meniscal tissue results frequently in the progressive development of osteoarthritris, involving degradation of joints, including the articular cartilage and sub-chondral bone. Osteoarthritis, a painful, debilitating condition is the most common form of arthritis. Osteoarthritis is incurable. Treatment options such as physiotherapy and pain medication are known to be effective at early stages or in milder forms of the disease. However, osteoarthritis is a degenerative process and in severe cases surgical procedures such as knee or hip replacement are required to offer some relief for the patient. Osteoarthritis is very common and the leading cause of chronic disability in the United States. Estimates suggest that in 2005 approximately twenty-seven million Americans suffer from osteoarthritis, and one in 2 people will develop osteoarthritis at some stage during their lifetime.

Full or partial meniscectomy has serious drawbacks which have shifted research interest towards the fields of biomaterials and bioengineering. Tissue engineering offers new treatment modalities and reduces side effects such as rejection of the donor organ and dependence of immune suppressive drugs. Creating three-dimensional (3D) bioscaffolds, however, is problematic as cells in culture usually migrate to form a two-dimensional layer and bioscaffolds are required to serve as 3D platforms. Current methods for creating 3D bioscaffolds such as particle leaching, gas forming, 3D printing or fused deposition modelling produce 3D structures, but they suffer from poor control of inner structure or resolution.

WO2012/054195 discloses a bio-printer for depositing cells and support material and so forming a construct with a defined geometry. Using this method the applicants fabricated multi-layered vascular tubes. WO2013/040087 discloses a platform for engineered, bioscaffold-free implantable tissues and organs and methods of making the same. The applicants disclose the fabrication of skeletal muscle tissue which can be maintained in culture.

Although tissue engineering methods have developed substantively, current techniques for the repair of meniscus tears or replacement of whole menisci by a tissue-engineered construct using bioscaffolding technologies such as synthetic polymers, hydrogels, ECM components, or tissue-derived materials, even with cell augmentation techniques have yet to yield sustained, reliable long-term results. The ideal meniscus construct is required to excel criteria such as mechanics (loading and lubrication), bioactivity (maintenance of cell phenotype, lack of immunogenicity, host tissue integration) and logistics (supply of artificial grown menisci, practical surgical implantation).

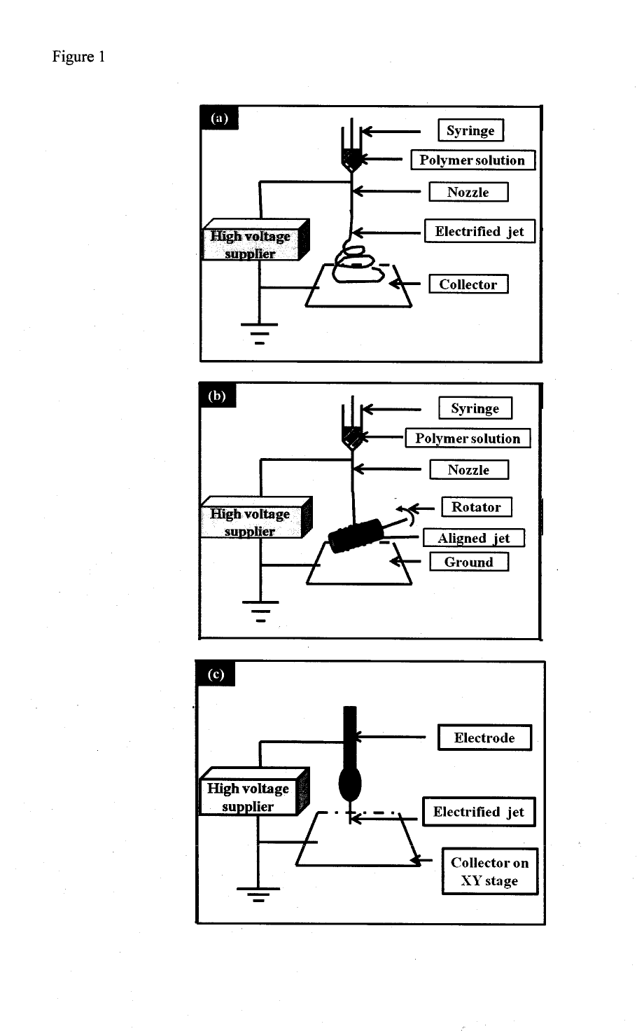

Biodegradable polymeric bioscaffolds have been widely used in tissue engineering as a platform for cell proliferation and subsequent tissue regeneration. Conventional micro-extrusion methods for 3D bioscaffold fabrication are, however, limited by their low resolution. Electro-spinning, a form of electro-hydrodynamic printing, is an attractive method to use due to its ability to fabricate high resolution bioscaffolds at the nanometer/micrometer scale level. With the assistance of an electric field between the nozzle and collector (FIG. 1a), the liquid in the nozzle is charged and stretched out. Subsequently, the jet is bent by the applied electrical force, and deposited randomly on the collector. The resultant micrometer/nanometer filaments are usually disordered, and pore sizes are less than 20 μm. In addition, the electro-spun meshes are in non-woven form, which are only useful for relatively small number of applications such as filtration. Researchers have made efforts to modify the process through the manipulation of electric field or the use of a dynamic collector, as shown in FIG. 1b, using a mandrel as the filament collector to achieve aligned filaments. A mat with most filaments aligned in one direction is achieved, and cells are seen to attach along the prevailing filament direction. However, the precise control of each filament orientation is not achieved. Furthermore, another significant drawback for both the conventional electro-spun meshes and bioscaffolds is the limited pore size, thus resulting in a slow rate of cell propagation. As reported in most of the in-vitro studies, the attached cells only formed a single layer on the top of the electro-spun bioscaffolds, and little evidence suggests that cells can grow into the depth of the bioscaffolds since the pore sizes are relatively small as compared to the size of the cells. Other workers have demonstrated the ability of a near-field electro-spinning process (NFES) (FIG. 1c), which can control the filament orientation during printing. With the help of a collector's movement, via a stage controller, filaments are deposited along the x- and y-axes. Although some initial attempts to fabricate 3D polymeric bioscaffolds using NFES have been performed, work has always been limited to 2D patterning due to the difficulty of solvent evaporation, and no 3D structures can be built so far. Quick solidification of the filaments over a very short distance between the nozzle and collector is normally required to build 3D structures, and this is certainly very challenging for solvent-based process. Electro-hydrodynamic hot jet plotting technique also has been used to fabricate high resolution (sub—10 μm) 3D bioscaffolds. However, high temperature is needed to melt the polymers during the fabrication process, thus limiting the application to materials that are temperature-sensitive (i.e. collagen, growth factors etc.) or materials that have high melting points.

In this study, we have developed an alternative electro-hydrodynamic jet printing (termed herein E-jetting) technique and employed it to fabricate 3D biodegradable polyester bioscaffolds, such as polycaprolactone (PCL) bioscaffolds, with the desired filament orientation and pore size. Results showed that solidified filaments were achieved at concentration >70% w/v and uniform filaments of diameter 20 μm were produced via the E-jetting technique, moreover, X-ray diffraction (XRD) and attenuated total reflectance fourier transform infrared (FTIR) spectroscopic analyses revealed that there were no physicochemical changes towards the biodegradable polyester, PCL. Bioscaffolds with a pore size of 450 μm and porosity level of 92%, were achieved. Further, a preliminary in-vitro study illustrated that live chondrocytes were attaching on the outer and inner surfaces of collagen-coated E-jetted PCL bioscaffolds. E-jetted bioscaffolds increased chondrocytes extra cellular matrix (ECM) secretion, and the newly-formed matrices from chondrocytes contributed significantly to the mechanical strength of the bioscaffolds. All these results show that E-jetting is an alternative bioscaffold fabrication technique, which has the capability to construct 3D bioscaffolds with aligned filaments and large pore sizes for tissue engineering applications.

### STATEMENTS OF THE INVENTION

According to a first aspect of the invention there is provided an apparatus for manufacturing a bioscaffold comprising: a positive pressure reservoir which, in use contains a polyester solution, and which is further in fluid communication with a nozzle from which said solution exits; a stage positioned adjacent said nozzle and adapted for movement along three axes X, Y and Z with respect to said nozzle and which, in use, supports a substrate on which said solution is deposited; and a voltage supply for creating an electric field between said nozzle and said stage or said substrate whereby solution exiting said nozzle flows as a continuous filament.

In a preferred embodiment of the invention said polyester solution is either biodegradable or non-biodegradable. Moreover, said polyester solution may comprise a natural polymeric material such as, but not limited to, collagen.

In a further preferred embodiment of the invention said reservoir is maintained at a positive pressure of between 0-400 kPa and most ideally between 150-250 kPa including all 1 kPa intervals there between, more ideally still a positive pressure of 200 kPa is used when operating the apparatus. Ideally, the pressure is maintained at a constant level when the bioscaffold is being manufactured. In a preferred embodiment said reservoir also includes a negative pressure device for exerting a negative pressure for use in instances where the polymer solution is to be retracted. More preferably said positive or negative pressure device is provided by a pneumatic arrangement, as is known by those skilled in the art.

In yet a further preferred embodiment of the invention said nozzle has an internal diameter between 80-510 μm and most ideally between 100-300 μm including all 1 μm intervals there between, more ideally still an internal diameter of either 500 μm or 200 μm is used when operating the apparatus.

In yet a further preferred embodiment of the invention the stage is positioned below said nozzle. Moreover, ideally the stage is positioned close to said nozzle i.e. at a distance between 1-10 mm including all 1 mm intervals there between, ideally at 2 mm. Further, ideally the stage is adapted to move along three axes X, Y and Z whilst said nozzle remains still. Alternatively, said nozzle is adapted to move along three axes X, Y and Z whilst said stage remains still. Alternatively, again, said stage is adapted to move along at least one of said three axes X, Y and Z and said nozzle is adapted to move along the remaining of said three axes X, Y and Z.

Reference herein to axes X, Y and Z is to left/right, backwards/forwards and up/down.

Preferably said stage is provided with either, or both, positioning members and securing members. The positioning members enable a substrate placed upon said stage to be positioned having regard to said nozzle and, ideally, the start position of said nozzle, and the securing members ensure, once positioned, the substrate is held in place during the bioscaffold fabrication process.

More preferably still said voltage supply enables a user to apply a voltage of between 0-20 Kv and most ideally between 1.5-5 kv including all 0.1 kv intervals there between, more ideally still a voltage of 2.2 kv is applied when operating the apparatus.

In a preferred apparatus of the invention said reservoir contains a biodegradable polyester solution such as Polycaprolactone (PCL), however, other biodegradable polyester solutions may be used such as poly(ethylene oxide), polyglycolide, poly(L-lactic acid), or poly(lactide-co-glycolide). Ideally said biodegradable polyester is dissolved in organic solution, preferably, at a concentration of between 30-80% w/v. Most typically concentrations greater than 40% are preferred, and most typically greater than 50% or 60%. Ideally, concentrations greater than 65% are preferred and, in increasing order of preference, 66%, 67%, 68%, 69%, 70%, 71%, 72%, 73%, 74%, 75%, 76%, 77%, 78%, 79%, and 80% w/v.

Ideally the organic solvent is acetic acid but other solvents such as formic acid, chloroform, dimethylformamide, methanol, or hexafluoroisopropanol can be used.

In one preferred use of the invention, said substrate is moved with respect to said nozzle in a back and forth manner whereby a first layer of said solution is deposited. Then said substrate is moved with respect to said nozzle away from same whereby the distance between said nozzle and said substrate is increased after which said substrate is moved with respect to said nozzle again in a back and forth manner whereby a second layer of said solution is deposited. Typically, layering of first and second layers is repeated until a desired depth of bioscaffold is produced. Those skilled in the art will appreciate that the precise movement of said substrate with respect to said nozzle can be automatically controlled so that 3D bioscaffolds of various sizes and shapes can be produced.

In yet a further preferred embodiment of the invention, said apparatus comprises a temperature control device whereby the temperature of the deposited or spun polyester can be controlled.

Accordingly, the apparatus of the invention further comprises a computer readable medium having computer executable instructions for performing the layering method comprising a program stored on a computer readable medium and adapted to be executed by a processor wherein said program performs the following functions:
a) moves said substrate with respect to said nozzle in a back and forth manner whereby a first layer of said solution is deposited;
b) moves said substrate with respect to said nozzle away from same whereby the distance between said nozzle and said substrate is increased; and
c) moves said substrate with respect to said nozzle in a back and forth manner whereby a second layer of said solution is deposited;
d) optionally, repeats steps a)-c) until a 3D bioscaffold is completed.

In a preferred method of the invention step b) further involves rotating said substrate with respect to said nozzle before performing step c). Ideally the degree of rotation is 90° but it may be any selected degree of rotation having regard to the structure of the biological material that the bioscaffold is being designed to mimic. For example the bioscaffold can be designed to mimic menisci, osteochondral tissue, tendons, ligaments or dentin.

In yet a further preferred method of the invention said back and forth movement may be along a straight or curved or zig-zaging or undulating line. Once again, the nature of the back and forth movement is selected having regard to the structure of the biological material that the bioscaffold is being designed to mimic.

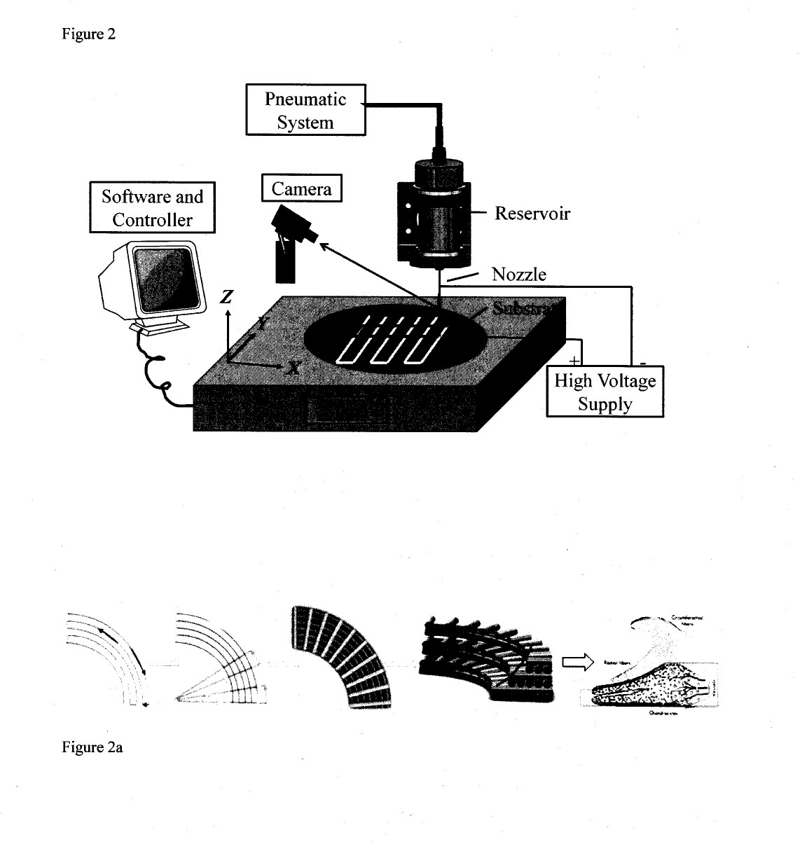

For example, in one embodiment of the invention, a bioscaffold for use to treat meniscal injuries is manufactured and the fabrication scheme for printing the meniscal bioscaffold is illustrated in FIG. 2a. The first layer is composed of semi-circles, such as for example ¼ circles, mimicking the circumferential fibres of the meniscus. Following the laying down of the first layer, a second layer which is composed of radial fibres, is laid on top of the curved fibres. By repeating these previous two steps, a multi-layer structure can be built up. To mimic the wedge-shaped meniscus, the number of circumferential fibres for each layer is decreased progressively.

According to a further aspect of the invention there is provided a data carrier comprising a program for executing the layering method of the invention.

According to a second aspect of the invention there is provided a method for manufacturing a bioscaffold comprising:
i) supplying under positive pressure a polyester solution;
ii) causing said solution to exit a nozzle and be deposited on a substrate;
iii) creating an electric field between said nozzle and said substrate whereby said solution exiting said nozzle flows as a continuous filament; and
iv) moving said substrate with respect to said nozzle along at least one of three axes X, Y and Z whereby said filament is laid upon said substrate in a selected manner to create a three-dimensional bioscaffold.

In a preferred method of the invention said substrate is moved with respect to said nozzle in a back and forth manner whereby a first layer of said solution is deposited.

In yet a further preferred method of the invention said substrate is moved with respect to said nozzle away from same whereby the distance between said nozzle and said substrate is increased.

In yet a more preferred method of the invention after said substrate is moved away said nozzle it is moved again in a back and forth manner whereby a second layer of said solution is deposited.

In a preferred method of the invention after the deposition of said first layer and either before or after the movement of said nozzle away from said substrate, said substrate is further rotated with respect to said nozzle, then said second layer is deposited. Ideally the degree of rotation is 90° but it may be any selected degree of rotation having regard to the structure of the biological material that the bioscaffold is being designed to mimic.

In yet a further preferred method of the invention said back and forth movement may be along a straight or curved or zig-zaging or undulating line. Once again, the nature of the back and forth movement is selected having regard to the structure of the biological material that the bioscaffold is being designed to mimic.

In a preferred method of the invention said layering of first and second layers is repeated until a desired depth of bioscaffold is produced. Those skilled in the art will appreciate that the precise movement of said substrate with respect to said nozzle can be automatically controlled so that 3D bioscaffolds of various sizes and shapes can be produced.

According to a further aspect of the invention there is provided a 3D bioscaffold comprising: a plurality of filamentous layers made from a polyester wherein the diameter of said filaments is between 3-50 μm including all 1 μm intervals there between, and the thickness of the bioscaffold is between 200-5000 μm including all 1 μm intervals there between.

In a preferred embodiment of the invention said diameter of said filaments is ideally between 3-50 μm, and ideally between 10-30 μm, most preferably 20 μm.

In yet a preferred embodiment of the invention said thickness of said bioscaffold is ideally between 200-5000 μm, and ideally between 300-5000 μm, most preferably greater than 500 μm where menisci is being made. Those skilled in the art will appreciate that the thickness of the bioscaffold will be determined by the nature of the biological material that the bioscaffold is to mimic.

In yet a preferred embodiment of the invention said 3D bioscaffold has a pore size between 100-500 μm, and ideally between 200-500 μm including all 1 μm intervals there between and most preferably of or about 250 μm.

In yet a preferred embodiment of the invention said 3D bioscaffold has a porosity between 70-95% and ideally between 80-95% including all 1% intervals there between and most preferably of or about 90%.

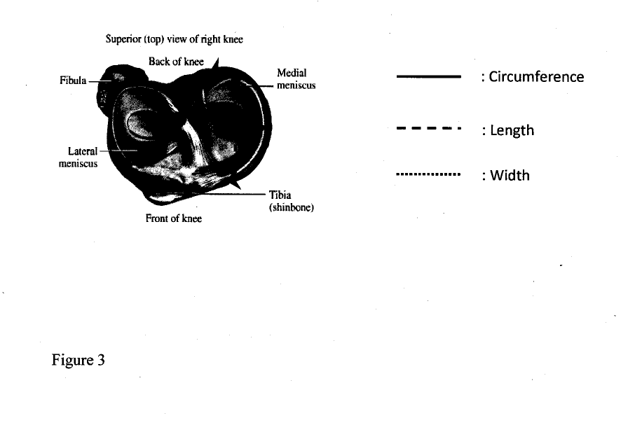

Preferably, the said 3D bioscaffold is sized and shaped in a bespoke manner so as to fill a defined cavity. More ideally still it is sized and shaped for use in meniscus repair. The dimensions of the natural meniscus are shown in Table 1 according to the diagram in FIG. 3. The size of the fabricated bioscaffold will be customized according to a patient's meniscus. Described herein is a meniscus manufactured according to the invention where its fibre diameter is 18.6±2.8 μm, and its pore size is 360±35 μm. In this embodiment of the invention said 3D bioscaffold is used in association with articular cartilage cells or cells with the potential to produce same such as progenitor cells or stem cells.

Thus the invention concerns the use of a 3D bioscaffold in the manufacture of an implant to treat injury or disease, particularly but not exclusively meniscal injury. In further embodiments the invention can be used to treat osteochondral, ligament, tendon, dentin, blood vessel and skin damage or disease.

Those skilled in the art will appreciate that the present invention can be used to create biodegradable and biocompatible 3D bioscaffolds including a variety of biomaterials, such as cells and growth factors, and having a complexly sculptured internal microstructure, that is designed to mimic a normal tissue's property.

Besides fabricating complex 3D bioscaffolds, this invention is believed to have utility in the coating and patterning of medical implants, moreover, it can also be used for dissolving drugs in the polymer solution so that when used as an implant the drugs are dispensed within and throughout the bioscaffold and/or medical implant.

The present invention provides the advantages of:
1. capability to process biocompatible materials without material denaturalization during fabrication;
2. capability to produce high resolution and well orientated micrometer-size fibers (with potential to nanometre-scale);
3. precise control of fibers both in spatial pattern and orientation, allowing the fabrication of biomimetic 3D tissue engineering bioscaffolds;
4. potential to build multi-material 3D bioscaffolds of complex microstructure and varied porosity level, in a single fabrication run at room temperature.

In the claims which follow and in the preceding description of the invention, except where the context requires otherwise due to express language or necessary implication, the word “comprises”, or variations such as “comprises” or “comprising” is used in an inclusive sense i.e. to specify the presence of the stated features but not to preclude the presence or addition of further features in various embodiments of the invention.

All references, including any patent or patent application, cited in this specification are hereby incorporated by reference. No admission is made that any reference constitutes prior art. Further, no admission is made that any of the prior art constitutes part of the common general knowledge in the art.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

Throughout the description and claims of this specification, the singular encompasses the plural unless the context otherwise requires. In particular, where the indefinite article is used, the specification is to be understood as contemplating plurality as well as singularity, unless the context requires otherwise.

An embodiment of the present invention will now be described by way of example only with particular reference to the following wherein:

FIG. 1 shows schematic diagrams of a (a) conventional electrospinning process, (b) modified electrospinning process with rotated collector, and (c) near-field electrospinning process.

FIG. 2 shows a schematic overview of the E-jetting system.

FIG. 2a shows a fabrication scheme for printing meniscal bioscaffold.

FIG. 3 shows a cross-sectional view of a meniscus, particularly showing the length, width and circumference referred to in Table 1.

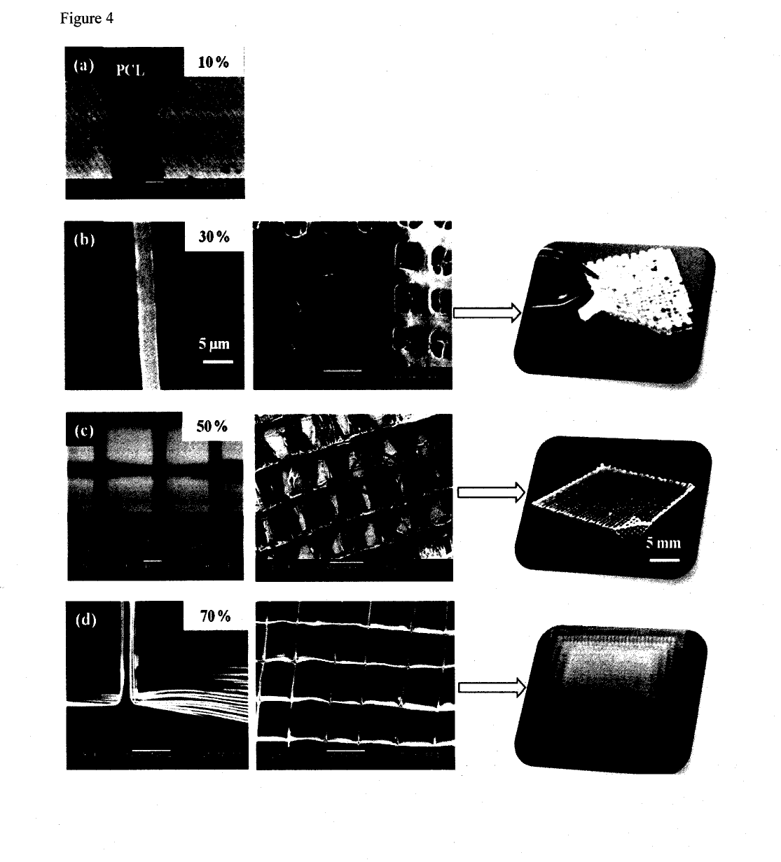

FIG. 4 shows printed results with various concentration of PCL solution (a) 10%, (b) 30%, (c) 50%, and (d) 70%.

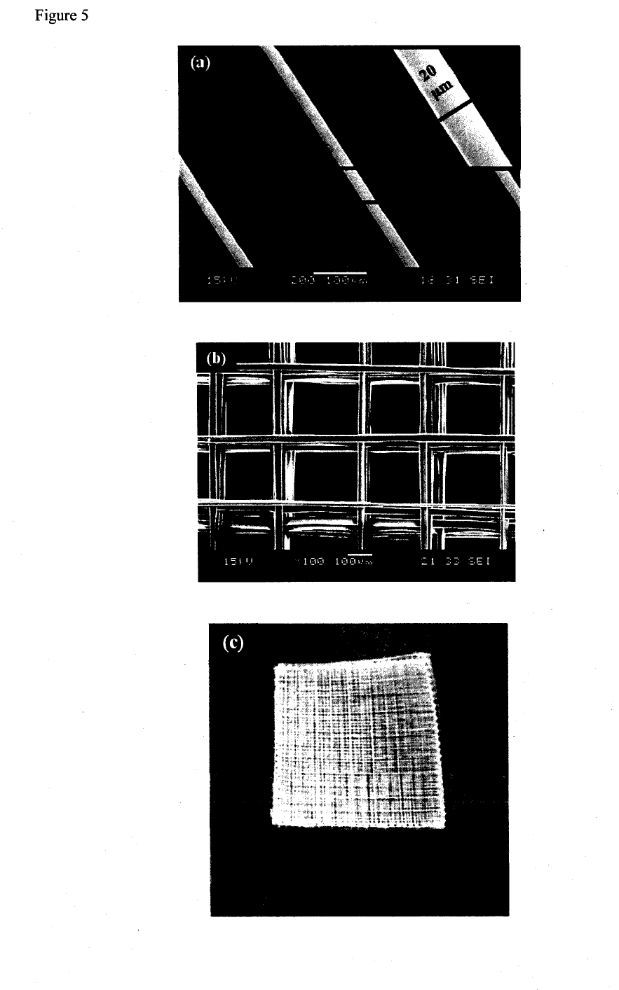

FIG. 5 shows highly porous bioscaffold fabricated via E-jetting technique. SEM image of the (a) E-jetted orientated filaments and (b) bioscaffold, (c) Snapshot of the bioscaffold.

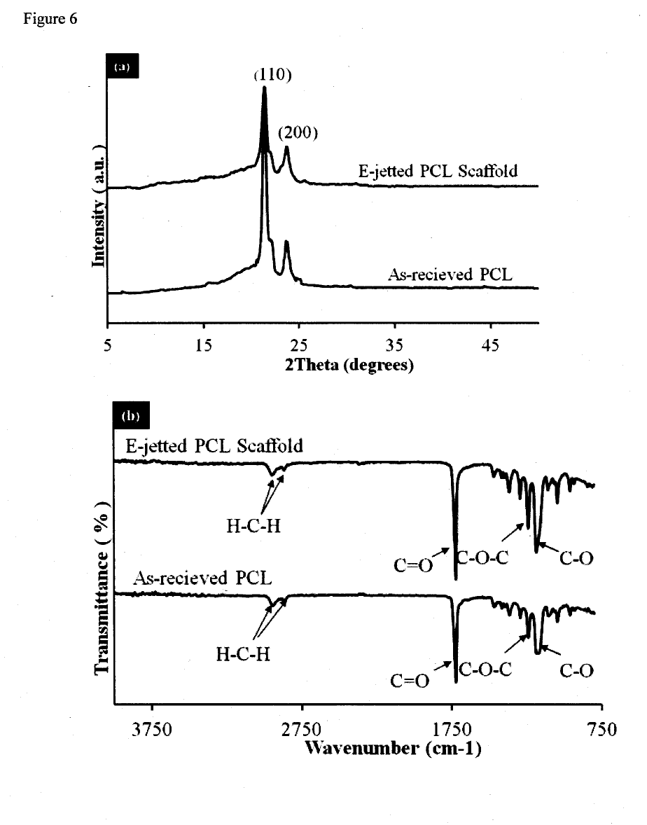

FIG. 6 illustrates the physicochemical characterizations of purchased PCL, and E-jetted PCL bioscaffold (a) XRD patterns, and (b) FTIR spectra.

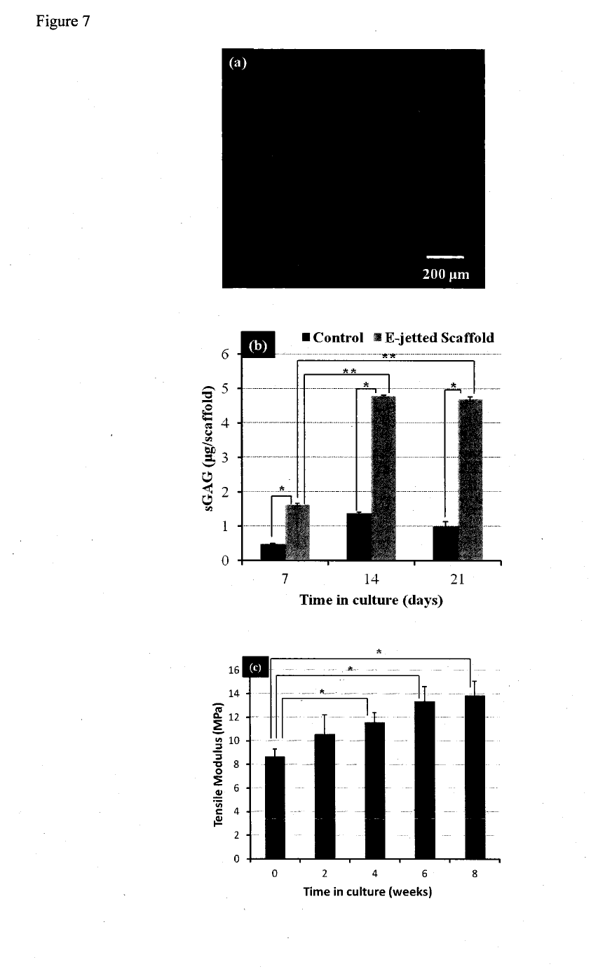

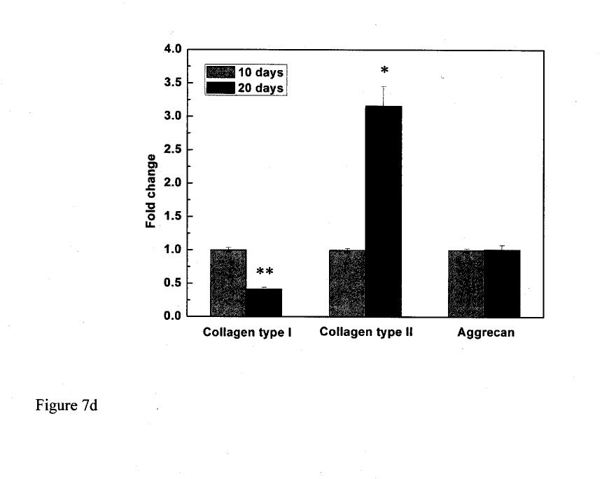

FIG. 7 shows chondrocytes responses on bioscaffolds (a) CLSM image of live/dead staining of chondrocytes on the fibrous bioscaffold, (b) sulfated glycoaminoglycan (sGAG) production on E-jetted bioscaffolds and control group (* p<0.05, ** p<0.05), (c) Tensile modulus of acellular bioscaffolds (0 week) and cell-laden bioscaffolds (2, 4, 6 and 8 weeks) (* p<0.05), (d) the expression of collagen type II and collagen type I production after culturing for 20 days reveals that the cells maintain their normal phenotypes as healthy chondrocytes in these bioscaffolds.

### MATERIALS AND METHODS

### Materials

PCL pellets with an average molecular weight of 80 kDa, and acetic acid (99.7% purity) were used in this study. Solutions of various weight volume ratios (w/v, PCL:acetic acid) ranging from 10 to 70%, were prepared by dissolving PCL pellets in acetic acid, and stirred continuously for 4 h to obtain a homogeneous PCL solution. This solution was then used for bioscaffold fabrication. Polished silicon wafers of diameter 100 mm were used as the substrates. These substrates were cleaned using ethanol pad, and left to air-dry prior to usage.

### E-Jetting Process

During the E-jetting process (FIG. 2), sufficient PCL solution was added into the reservoir fitted with a 500 μm inner diameter stainless steel nozzle, which was positioned above the silicon wafer (nozzle-substrate distance of 2 mm), and the substrate was placed on the XYZ stage. Positive pressure of 200 kPa was used in providing a constant solution supplement, and high direct current (DC) voltage of 2.2 kV was applied between the nozzle and substrate to charge the solution, thereby creating filaments continuously.

By increasing the electrical field, the droplet at the tip of the nozzle was elongated, and thin jet formed until the surface tension of the droplet was overcome. As demonstrated in FIG. 3, the orientation of filaments was achieved by the movement of XYZ stage, and continuous filaments were placed along the Y-axis to form the first layer with a back-and-forth motion. Similarly, the second layer was achieved along the X-axis, and the above procedure was repeated with the needle moving upward along the Z-axis until the bioscaffold was built with a pre-defined number of layers.

### Bioscaffold Characterization

The filament's diameter was measured using an atomic force microscope (AFM) (SPM5, Seiko Instruments), at a scanning frequency of 0.1 Hz. Morphology of the E-jetted PCL bioscaffolds was studied using a scanning electron microscope (SEM), operating at an accelerating voltage of 15 kV and current of 10 mA. Crystallinity of the as-received PCL and E-jetted PCL bioscaffolds was determined using X-ray diffraction (XRD). A diffractometer CuK_{α} radiation was used, operating at 40 kV and 30 mA. Data was collected over a 2θ range of 5-50° with a step size of 0.05° and a count time of 20 s. Attenuated total reflectance fourier transform infrared (FTIR) spectroscopic analysis of as-received PCL and E-jetted PCL bioscaffolds was performed over a range of 800-4000 cm⁻¹ at a resolution of 8 cm⁻¹, averaging 64 scans. To obtain the porosity of the E-jetted bioscaffolds, three bioscaffolds of pore size 500 μm were fabricated with 20 layers of filaments printing. All samples were weighed with an electronic balance (±0.1 mg), and the dimensions of the samples were measured with a micrometer (±1 μm). The porosity of the bioscaffolds was then calculated using the following equation:

$$\begin{matrix}{V}_{s} = L \times W \times H & ( 1 ) \\ {ρ}_{s} = \frac{{M}_{s}}{{V}_{s}} & ( 2 ) \\ Porosity ( \% ) = ( 1 - \frac{{ρ}_{s}}{{ρ}_{PCL}} ) \times 100 \% & ( 3 )\end{matrix}$$

where Mₛ and Vₛ are the mass and volume of the bioscaffold; L, W and H are the length, width and height of the bioscaffold; ρₛ is the density of bioscaffold; ρ_{PCL} (1100 kg/m³) is the density of PCL.

### Meniscus Features

The meniscal bioscaffold was constructed with circumferential PCL/collagen fibers interspersed with radial PCL/collagen fibers, mimicking the internal microstructure of the normal meniscus. The diameter of the fibers was 18.6±2.8 μm, which was comparatively smaller than those fabricated using a micro-extrusion system having a fiber diameter of 100 μm. Fine fibers have been shown to enhance cell attachment and modulate cell signaling pathways, thereby accelerating extracellular matrix production. The pore size of the bioscaffold was 360±35 μm, which was in the desirable range to provide sufficient blood and nutrient transfer within the bioscaffold, though the mechanical strength of the bioscaffold would be compromised.

### In-Vitro Study

Porcine chondrocytes were harvested from the knee joint of 1 year pig, and cultured in Dulbecco's modified eagle's medium (DMEM) supplement with 10% fetal bovine serum, 2% L-Glutamine, and 1% penicillin/streptomycin. The cells were incubated at 37° C. with 5% carbon dioxide atmosphere. Medium was changed every 2 days. Cells were detached and re-suspended in DMEM until 70-80% cell confluence was achieved.

Sterilization of the E-jetted bioscaffolds using ultraviolet (UV) light was carried out for 15 min. The bioscaffolds were then immersed in 2 mg/ml solution of dopamine (10 mM Tris buffer, pH 8.5) overnight in the dark, followed by rinsing with ultrapure water to remove the un-attached dopamine. Collagen grafting on the polydopamine-coated PCL bioscaffolds was done with collagen (0.1 M in acetic acid) and incubated overnight in a humid atmosphere at 37° C. The bioscaffolds were washed twice with sterile phosphate buffer saline (PBS) solution to remove un-attached collagen, and left to air-dry in a sterile environment prior to cell seeding. Bioscaffolds were then placed into a 24-well plate, and 50 μl of cell aliquot was seeded at the density of 4×10⁵ cells/cm². Cells seeded on 24-well polystyrene culture dishes (1×10⁵ cells/cm²) were used as controls. The cell-seeded bioscaffolds and control were kept at 37° C. in an incubator for 4 h for cell attachment before transferring to a new 24-well plate, and 1 ml medium was added. Medium was changed every 2 days during the 8-week cell culturing.

To visualize the population of the live and dead chondrocytes, the bioscaffolds were stained with calcein and ethidium bromide after 3 days of culturing. For cell survival examination, Dulbecco's phosphate buffered saline (DPBS) solution with 2 μmol/l of acetomethoxy derivate of calcein (Calcein-AM) and 2 μmol/l of ethidium homodimer-1 (EthD-1) (LIVE/DEAD Viability/Cytotoxicity Kit, Invitrogen) was used to incubate chondrocytes/bioscaffolds for 1 h. Calcein-AM exhibits green fluorescent in live cells, and EthD-1 presents as red fluorescent in dead cells. The chondrocytes/bioscaffolds were then observed using an inverted epifluorescence microscope.

The measurement of sulfated glycoaminoglycan (sGAG) production was conducted to examine whether cells were functional and able to produce cartilage-like extracellular matrix (ECM). The cell-seeded bioscaffolds and control were taken out at culture day 7, 14 and 21, washed twice with PBS and digested in 0.5 ml of papain extraction reagent overnight at 65° C. in a water bath. Then, the total sGAG production was determined using the Blyscan kit (Biocolor, UK). The procedures were carried out according to the manufacturer's protocol. Absorbance was measured at 656 nm using the microplate reader.

Tensile test of acellular and cell-laden bioscaffolds at week 2, 4, 6 and 8 was carried out using a table top tensile tester (Instron 3345, Canton, Mass.), at a load cell capacity of 100 N, and samples were extended to failure at a rate of 1 mm/min. Cross-sectional area and gauge length of the bioscaffolds were determined by measuring the width and thickness using a micrometer. Using the cross-sectional area and gauge length, tensile modulus was calculated from the stress-strain curve. Three replicates were measured, and the mean value was calculated.

### Statistical Analysis

A t-test was used to determine any significant differences existed between the mean values of the experimental groups. A difference between groups was considered to be significant at p<0.05.

### EXAMPLE 1

### Effect of PCL Solution Concentration

Concentration, here referring to the weight volume ratio of PCL in acetic acid, is critical for the filament formation, and thus bioscaffolds fabrication. In traditional electro-spinning process, the most commonly used PCL solution concentration was 8-12% (Seeram et al. 2005) whilst in E-Jetting process, there was no filament being generated at such a low concentration (<10%) (FIG. 4a). There only appeared a wide line of solution on the substrate, attributing to the insufficient time for solvent evaporation. The printed line took more than 5 min to dry out completely, and thus it was not possible to build up a bioscaffold architecture. Increasing the PCL solution concentration to 70% was studied to investigate the concentration effect on filament generation and bioscaffold fabrication, and the results were illustrated here.

It was able to obtain PCL filaments using 30% solution. Repeated printing of 200 layers alternately along the x- and y-axes could also be done. However, after detaching the bioscaffold from the substrate, the sample obtained was only a thin film with thickness of 47±7 μm. All the filaments on the same location tend to merge together, and could not discern clearly from each other. Furthermore, after printing few layers, undesirable auxiliary filaments were generated as shown in FIG. 4b (enclosed in inset square). The accompanying tiny filaments were attributed to the increased electric field contributed by the accumulation of conductive solvent on the substrate. It revealed that there was still substantial amount of solvent entrapped within the filaments.

With increasing concentration to 50%, PCL filaments were successfully generated, and the diameter of the resultant filaments was 3.1±0.1 μm, which was measured using an AFM. After printing 200 layers, a thickness of 246±37 μm was achieved, demonstrating an improved performance with increasing concentration. However, the filaments still had a tendency to merge together. From FIG. 4c, it was found that the filaments collapsed (enclosed in inset circle), owing to insufficient evaporation time for the solvent. Quick solidification was critical for the filaments to span over the designed gap (500 μm) underneath them for 3D construction.

As presented in FIG. 4d, it was observed that uniform single filaments were achieved at 70% concentration, and a 3D bioscaffold was successfully built up after printing 50 layers. The thickness of the bioscaffold was 965±19 μm. The filaments did not merge together along the same axis, implying that the filaments solidified quickly upon ejecting. However, the diameter of the filaments was 20.2±0.9 μm, which was comparatively larger than that of 50% concentration. This phenomenon was attributed to the change in solution properties, which included electric conductivity, surface tension, viscosity and solvent evaporation rate. These properties determined the amount of solution being stretched out from the tip of the nozzle under high voltage, thus affecting the resultant filament morphology. Higher electrical conductivity was necessary during the process initiation to overcome the surface tension of the solution, and also important in the subsequent stretching of the electro-hydrodynamic jet. With increasing PCL concentration, less acetic acid would be present in the solution and thus, fewer free ions, which led to a decrease in electric conductivity, but, the viscosity and surface tension were increased, thereby resulting in larger filament's diameter. However, the solvent evaporation rate was improved since there was less acetic acid in the solution and rapid solidification of the filaments could be achieved towards 3D construction. In this study, all other processing parameters remained unchanged such as positive pressure, voltage and moving speed of the stage, while all these parameters could affect the feed rate or stretching of the solution during printing. The filament diameter could be decreased by decreasing the solution feed rate, increasing the high voltage potential or increasing the moving speed of the stage.

For E-jetting process, quick solidification of the filaments over a very short distance (2 mm) between the nozzle and collector was achieved with increasing PCL concentration up to 70%. Fibrous bioscaffolds were successfully fabricated with multi-layers of specific aligned filaments and controlled pore size of 450±50 μm. Compared with the electro-spun bioscaffolds, E-jetted bioscaffolds could provide sufficient space for nutrient and blood transfer, promoting cell growth within the 3D bioscaffold. It was demonstrated that E-jetting technique had the capability to construct 3D bioscaffolds with desirable pore size and filament orientation.

### EXAMPLE 2

### Characterization of Bioscaffolds

SEM examination of the E-jetted PCL filament is shown in FIG. 5a. Parallel filaments were obtained, and the surface of the printed filaments was generally smooth. With the attribution of high voltage, uniform filaments of diameter 20.5±1.9 μm were achieved. It was comparatively smaller than those fabricated using a micro-extrusion system having a filament diameter of 100 μm (Kalita et al. 2003, Wei et al. 2012). Fine filaments have shown to enhance cell attachment and modulate cell signalling pathways, thereby accelerating extracellular matrix production (Nur-E-Kamal et al. 2005, Li et al. 2006).

As reported in the literature, the pore sizes of electrospun bioscaffolds were relatively small as compared to the size of cells, thus limiting cell penetration into the bioscaffolds (Kidoaki et al. 2005). To evaluate this, a 10-layered bioscaffold was fabricated in this study (FIG. 5b). The pore size obtained was 450±50 μm. The large pore sizes would certainly be able to support and guide cell in-growth, restricting cell colonisation on the outer surfaces only. With continuous printing to 50 layers, a highly porous PCL bioscaffold of dimensions 15 by 15 mm was fabricated with a porosity level of 92±3% (FIG. 5c). The whole fabrication process took approximately 40 min. High porosity level of up to 90% was desirable in providing sufficient blood and nutrient transfer within the bioscaffold (Agrawal and Ray 2001), though the mechanical strength of the bioscaffold would be compromised.

As shown from the XRD patterns (FIG. 6a), the diffraction of (110) and (200) lattice planes of the semi-crystalline PCL appeared as strong peaks at 28=21.5 and 23.8°. The intensity ratio (I100/I200) (Lee et al. 2003) of the two main peaks of E-jetted PCL bioscaffold was 2.4, which was lower than that of the as-received PCL (I100/I200=3.6). This implied that the retardation of crystallization occurred in the filaments, revealing that there was no elongation of PCL chains during the E-Jetting process.

Characteristic C═O peak at 1723 cm⁻¹, CH₂ asymmetric stretching at 2945 cm⁻¹ and symmetric stretching at 2865 cm⁻¹, C—O—C stretching at 1241 cm⁻¹ and C—O stretching at 1170 cm⁻¹, all belonging to PCL were detected in the FTIR spectra for all samples (FIG. 5b), demonstrating that there was no destruction of PCL caused by the E-jetting technique.

### EXAMPLE 3

### In-Vitro Study of Chondrocytes Responses on Bioscaffolds

After culturing for 3 days, the viability of chondrocytes on the fibrous bioscaffolds was evaluated using live/dead staining. It was observed that there were numerous live chondrocytes (highlighted parts of the image) attaching and spreading on the surfaces of collagen-coated PCL filaments, showing good viability of cells (FIG. 7a). In addition, chondrocytes were seen adhering on the inner surfaces of the bioscaffold, demonstrating good cell infiltration. Some cells were well-focused in plane whilst others were out of focused, implying that these cells were attaching in different layers of the bioscaffold.

FIG. 7b shows the sGAG production on cell-laden bioscaffolds and control. The amount of sGAG content on each sample increased with culturing time. However, sGAG produced by chondrocytes on bioscaffolds was significantly higher than that of the control at all time points. Results revealed a two-fold increase in sGAG content on bioscaffolds as compared to control on day 14 and 21. This demonstrated that the chondrocytes maintained their normal phenotype and the E-jetted bioscaffolds increased chondrocytes ECM secretion, thus showing capability for tissue repair.

FIG. 7c presents the results of tensile test conducted on acellular E-jetted bioscaffolds (0 week) and cell-laden bioscaffolds (2, 4, 6 and 8 weeks). The tensile modulus of cell-laden bioscaffolds increased with culturing time, and the modulus of cell-laden bioscaffolds at week 4, 6 and 8 was significantly higher than that of the a-cellular bioscaffolds, demonstrating that newly-formed matrices from chondrocytes contributed significantly to the mechanical strength of the bioscaffolds. The tensile modulus of the cell-seeded bioscaffolds at week 8 was 13.9±1.2 MPa, was comparable to that of the human meniscus in radial direction, meaning the load transmitting, shock absorbing and joint stabilizing functions of the meniscus had been replicated. When replacing injured meniscus with a bioscaffold, similar mechanical properties are generally preferred. If not, the bioscaffold is expected to experience an unrecoverable deformation, thereby inducing cartilage wearing and increasing cartilage degeneration, which subsequently compromises knee function.

FIG. 7d shows the expression of collagen type II production after culturing for 20 days is about three folds higher than that after culturing for 10 days, whilst the expression of collagen type I production decreases by half. These results reveal that the cells maintain their normal phenotypes as healthy chondrocytes in these bioscaffolds.

### REFERENCES

Agrawal C M and Ray R B 2001 Biodegradable polymeric bioscaffolds for musculoskeletal tissue engineering J Biomed Mater Res 55 141-50.
Kalita S J, Bose S, Hosick H L and Bandyopadhyay A 2003 Development of controlled porosity polymer-ceramic composite bioscaffolds via fused deposition modeling Mat Sci Eng C-Bio S 23 611-20.
Kidoaki S, Kwon I K and Matsuda T 2005 Mesoscopic spatial designs of nano- and microfiber meshes for tissue-engineering matrix and bioscaffold based on newly devised multilayering and mixing electrospinning techniques Biomaterials 26 37-46.
Lee K H, Kim H Y, Khil M S, Ra Y M and Lee D R 2003 Characterization of nano-structured poly(epsilon-caprolactone) nonwoven mats via electrospinning Polymer 44 1287-94.
Li W J, Jiang Y J and Tuan R S 2006 Chondrocyte phenotype in engineered fibrous matrix is regulated by fiber size Tissue Eng 12 1775-85.
Nur-E-Kamal A, Ahmed I, Kamal J, Schindler M and Meiners S 2005 Three dimensional nanofibrillar surfaces induce activation of Rac Biochem Bioph Res Co 331 428-34.
Seeram R, Kazutoshi F, Wee E T, Teik C L and Zuwei M 2005 An Introduction to Electrospinning and Nanofibers: World Scientific Publishing Co. Pte. Ltd).

TABLE 1
Dimensions of medial and lateral meniscus in the human body

| | Length (mm) | Width (mm) | Circumference (mm) |
|---|---|---|---|
| Medial Meniscus | 40.5-45.5 | 27 | 90-110 |
| Lateral Meniscus | 32.4-35.7 | 26.6-29.3 | 80-100 |

1. An apparatus for manufacturing a bioscaffold comprising: a positive pressure reservoir which, in use contains a polyester solution, and which is further in fluid communication with a nozzle from which said solution exits; a stage positioned adjacent said nozzle and adapted for movement along three axes X, Y and Z with respect to said nozzle and which, in use, supports a substrate on which said solution is deposited; and a voltage supply for creating an electric field between said nozzle and said stage or said substrate whereby solution exiting said nozzle flows as a continuous filament.

2. The apparatus according to claim 1 wherein said reservoir is maintained at a positive pressure of between 0-400 kPa.

3. The apparatus according to claim 1 wherein said positive pressure reservoir is maintained at a constant pressure when the bioscaffold is being manufactured.

4. The apparatus according to claim 1 wherein said reservoir also includes a negative pressure device.

5. The apparatus according to claim 1 wherein said positive pressure reservoir or said negative pressure device comprises a pneumatic arrangement.

6. The apparatus according to claim 1 wherein said nozzle has an internal diameter between 80-510 μm.

7. The apparatus according to claim 1 wherein said stage is positioned below said nozzle.

8. The apparatus according to claim 1 wherein said stage is adapted to move along three axes X, Y and Z whilst said nozzle remains still.

9. The apparatus according to claim 1 wherein said stage is provided with either, or both, positioning members and securing members.

10. The apparatus according to claim 1 wherein said voltage supply enables a user to apply a voltage of between 0-20 Kv.

11. The apparatus according to claim 1 wherein said apparatus comprises a temperature control device.

12. The apparatus according to claim 1 wherein said apparatus further comprises a computer readable medium having computer executable instructions for performing a layering method comprising a program stored on a computer readable medium and adapted to be executed by a processor wherein said program performs the following functions:
a) moves said substrate with respect to said nozzle in a back and forth manner whereby a first layer of said polyester solution is deposited;
b) moves said substrate with respect to said nozzle apart whereby the distance between said nozzle and said substrate is increased; and
c) moves said substrate with respect to said nozzle in a back and forth manner whereby a second layer of said polyester solution is deposited;
d) optionally, repeats steps a)-c) until a 3D bioscaffold is completed.

13. The apparatus according to claim 12 wherein step b) further involves rotating said substrate with respect to said nozzle before performing step c).

14. The apparatus according to claim 12 wherein said back and forth movement is along a straight or curved or zig-zaging or undulating line.

15. A method for manufacturing a bioscaffold comprising:
i) supplying under positive pressure a polyester solution;
ii) causing said solution to exit a nozzle and be deposited on a substrate;
iii) creating an electric field between said nozzle and said substrate whereby said solution exiting said nozzle flows as a continuous filament; and
iv) moving said substrate with respect to said nozzle along at least one of three axes X, Y and Z whereby said filament is laid upon said substrate in a selected manner to create a three-dimensional bioscaffold.

16. The method according to claim 15 wherein said substrate is moved with respect to said nozzle in a back and forth manner whereby a first layer of said solution is deposited.

17. The method according to claim 15 wherein said substrate is moved with respect to said nozzle apart whereby the distance between said nozzle and said substrate is increased.

18. The method according to claim 17 wherein after said substrate is moved apart from said nozzle it is moved again in a back and forth manner whereby a second layer of said solution is deposited.

19. The method according to claim 17 wherein after the deposition of said first layer and either before or after the movement of said nozzle apart from said substrate, said substrate is further rotated with respect to said nozzle.

20. The method according to claim 19 wherein after said rotation a second layer is deposited.

21. (canceled)

22. The method according to claim 15 wherein said layering of first and second layers is repeated until a desired depth of bioscaffold is produced.

23-27. (canceled)

28. A bioscaffold manufactured according to the method of claim 15.

29-30. (canceled)

31. A 3D bioscaffold comprising a plurality of filamentous layers made from a polyester wherein the diameter of said filaments is between 3-50 μm and the thickness of the bioscaffold is between 200-5000 μm.

32-41. (canceled)
